(19) United States
(12) Patent Application Publication
(10) Pub. No.: US 2006/0039978 A1
(43) Pub. Date: Feb. 23, 2006

(54) Self-forming phospholipidic gels

(76) Inventor: Julia Eva Diederichs, Dusseldorf (DE)

Correspondence Address: GUDRUN E. HUCKETT DRAUDT, LONSSTR. 53, WUPPERTAL, 42289, DE

(21) Appl. No.: 10/534,746

(22) PCT Filed: Nov. 24, 2003
(86) PCT No.: PCT/DE03/03883

§ 371 Date: May 12, 2005

(30) Foreign Application Priority Data
Nov. 26, 2002 (DE) 102 55 285.1

(51) Int. Cl. A61K 9/14; A61K 31/56; A61K 31/192

(52) U.S. Cl. 424/484; 514/171; 514/569

(57) ABSTRACT

A phospholipid gel is spontaneously formed when combining a first phospholipid, a second phospholipid, and water, wherein the first phospholipid is a neutral phospholipid and the second phospholipid is a negatively charged phospholipid. The first and second phospholipids are of natural origin, semi-synthetic origin, or synthetic origin. Preferably, they are di(C₈-C₂₂ acyl) phosphatidyl choline and di(C₈-C₂₂ acyl) phosphatidyl glycerol. The total phospholipid concentration is within a range of 6-40% by weight.

The invention relates to self-forming gels comprised of natural, semi-synthetic and synthetic phospholipids and water.

The gels can be used as such for a moisturizing or calming treatment of skin, mucous membrane, natural or surgically generated body cavities or may contain pharmacologically active substances that are released on or into the skin, mucous membrane, natural or surgically generated body cavities or compartments. The gels can be added as a stabilizer or a solubilizer to pharmaceutical formulations.

Phospholipids are used in the form of liposomes as topical medicament carriers [Schreier & Bouwstra, J. Control. Release 30, 1-15, 1994; Cevc, Crit. Rev. Ther. Drug Carrier Syst. 13, 257-288, 1996; Yarosh, Photodermatol. Photoimmunol. Photomed. 17, 203-212, 2001] and as components of cosmetic preparations such as creams and lotions [Weiner et al., J. Drug Target. 2, 405-410, 1994]. Usually, liposomes are used directly in their aqueous dispersed form or are worked into a gel-forming matrix including pharmaceutically employed base creams or hydrogels.

However, several types of phospholipid gels and their corresponding preparation methods are disclosed. Ghyczy and co-workers [Ghyczy et al. EP 0514435 B1] describe an alcoholic phospholipid gel with a phospholipid contents of 15-30% and 14-20% alcohol. Three-dimensional liposome networks of highly concentrated (60%) semi-solid phospholipid dispersions have been developed and characterized by Brandl and coworkers. [Brandl et al., Adv. Drug Deliv. Rev. 24, 161-164, 1997; Brandl et al., Chem. Phys. Lipids 87, 65-72, 1997; Brandl et al. U.S. Pat. No. 6,399,094]. Vesicular phospholipid gels that are comprised of 40% phosphatidyl choline and cholesterol have been used as carriers for cytostatic agents for local treatment of cancer. [Moog et al., J. Liposome Res. 8, 87-88, 1998; Güthlein et al., J. Liposome Res. 10, 251-252, 2000; Unger et al., WO 99/49716]. Ibscher [Dissertation, Universität Freiburg, Germany, 2000; Ibscher & Fridrich, WO 01/13887 A2] has developed a phospholipid gel as a topical carrier for antiviral treatment of the skin that is comprised of phospholipid, alcohols and sugar alcohols or carbohydrates. Vesicular systems that are comprised of a minimal phospholipid content (2%) and a high alcohol content (30%), so-called ethosomes, are also described for topical application and for transport of active substances into the skin [Touitou et al., J. Control. Release 3, 403-418, 200; Dayan & Touitou, Biomaterials 21, 1879-1885, 200; Touitou, WO 95/35095].

In contrast to the above systems, it was surprisingly found that neutral and negatively charged phospholipids in low concentrations mixed in water spontaneously form gels that are stable enough in order to be processed further, for example, to be filled into containers or syringes and to be applied to human skin or body compartments. Moreover, the gels stabilize pharmaceutical formulations, i.e., sparingly soluble materials are maintained in solution and precipitation is prevented.

The object of the present intention is a phospholipid gel comprised of a neutral phospholipid and a negatively charged phospholipid and water.

The phospholipids employed in the gels of the present invention can be selected from natural, semi-synthetic or synthetic phospholipids.

The employed phospholipids according to the invention can be selected from natural, semi-synthetic and synthetic phospholipids. Suitable phospholipids that can be used in the phospholipid gel according to the invention are, for example, phosphatidyl cholines. Examples of natural neutral phospholipids are soy phosphatidyl choline and phosphatidyl choline derived from egg. As synthetic or semi-synthetic phospholipids, any fatty alkanoyl phosphatidyl choline, in particular, those derived from saturated or unsaturated C₈-C₂₂ alkanoyl phosphatidyl choline, can be used. The fatty alkanoyl groups are derived, for example, from caprylic acid, pelargonic acid, capric acid, undecanoic acid, lauric acid, tridecanoic acid, myristic acid, pentadecanoic acid, palmitic acid, margaric acid, stearic acid, nonadecanoic acid, arachic acid, behenic acid, tuberculostearic acid, palmitoleinic acid, oleic acid, erucic acid, linolic acid, linolenic acid, elaeostearic acid, arachidonic acid, clupanodonic acid, docosahexaenoic acid, and any mixture thereof. A preferredly employed phosphatidyl choline is dipalmitoyl phosphatidyl choline.

As negatively charged phospholipids those are particularly suitable that contain a “Abonsäuresalz” (translators's note: this is apparentlya typing error and should read “Carbonsäcuresalz” which translates to “carboxylic acid salt”) group in the molecule. Examples of negatively charged phospholipids are, for example, phosphatidyl glycerol that is a naturally occurring negatively charged phospholipid. Further examples are dialkanoyl phosphatidyl glycerol, wherein the alkanoyl group can be derived from the above-mentioned fatty acids. To be mentioned as examples of suitable dialkanoyl phosphatidyl glycerol are dipalmitoyl phosphatidyl glycerol and dimyristoyl phosphatidyl glycerol. As negatively charged phospholipids phosphatidyl serine and phosphatidyl acid are also suitable and can contain also fatty acid chains in the molecule; in this case, the fatty acid chains can be derived from the above-mentioned fatty acids, for example, derived from palmitic acid. A further negatively charged phospholipid is, for example, phosphatidyl inositol. The negatively charged phospholipids have as cationic counter ions, preferably, alkali ions or ammonium ions. The selection of cations is not limited to certain cations as long as they are physiologically compatible.

In the phospholipid gels according to the invention, the total phospholipid concentration is preferably in a range between 6 and 40% by weight. The ratio of neutral phospholipid to negatively charged phospholipid can be selected within wide ranges; preferably, the ratio of neutral phospholipid to negatively charged phospholipid is in the range of 10:0.01 to 10:5, in particular in the range of 10:1 to 10:0.25.

Pharmacologically active substances can be incorporated into the phospholipid gel according to the invention. Examples of the active substances are steroids, non-steroidal antiphlogistic agents, antibiotics, antioxidants, or antiepileptic agents. The steroids can be selected, for example, from the group comprised of hydrocortisone or dexamethasone; the non-steroidal antiphlogistic agent can be selected from the group comprised of ibuprofen, diclofenac, flurbiprofen or nabumetone; the antibiotic can be selected from the group comprised of tetracycline or one of its derivatives, an aminoglycoside, for example, gentamycine or neomycine, a macrolid antibiotic, for example, erythromycine, a nitroimidazole derivative, such as metronidazole or flucidic acid, an antibiotic peptide or an antibiotic oligonucleotide; the antioxidant can be selected from the group comprised of vitamin E or coenzyme Q₁₀; the antiepileptic agent can be selected from the group comprised of valproic acid and its salts.

As an example of a particularly suitable embodiment a mixture of a soy phospholipid choline and phosphatidyl glycerol is to be mentioned.

Neutral phospholipids in a concentration of, for example, 5 to 30%, in particular, of 5 to 20% and negatively charged phospholipids in a concentration of, for example, 0.25 to 10% form spontaneously a gel when they are mixed with water. This can be carried out, for example, with the natural components soy phosphatidyl choline and phosphatidyl glycerol but also with mixtures of synthetic phosphatidyl choline/phosphatidyl glycerol such as dipalmitoyl phosphatidyl choline and dipalmitoyl phosphatidyl glycerol ordimyristoyl phosphatidyl glycerol. The gel forms spontaneously from a thin lipid film when it is dispersed in water while being shaken gently.

Dispersion under great shearing forces and high pressure (high-pressure homogenization) is not required. Organic solvents, detergents, or bridge-forming bivalent ions are also not required. Active substances can be incorporated into the gel; in particular, substances of the ubichinone type such as coenzyme Q₁₀ can be present without disturbing the formation and stability of the gel structure.

### EXAMPLES

### Example 1

1A: 180 mg soy phosphatidyl choline and 20 mg egg phosphatidyl glycerol are deposited as a thin film on a glass wall. 1 ml distilled water is added and the container is shaken on a shaker at low speed until a gel has formed. The gel is transferred into a syringe and stored at 4° C.

1B: The same process is carried out in order to form a gel of 190 mg phosphatidyl choline and 10 mg phosphatidyl glycerol.

1C: The same process is carried out in order to form a gel of 195 mg phosphatidyl choline and 5 mg phosphatidyl glycerol.

1D: The same process is carried out in order to form a gel of 90 mg phosphatidyl choline and 10 mg phosphatidyl glycerol.

1E: The same process is carried out in order to form a gel of 360 mg phosphatidyl choline and 40 mg phosphatidyl glycerol.

### Example 2

2A: 150 mg dipalmitoyl phosphatidyl choline and 15 mg dimyristoyl phosphatidyl glycerol are deposited as a thin film on a glass wall. 1 ml distilled water is added and the container is shaken on a shaker at low speed until a gel is formed. The gel is transferred into a syringe and stored at 4° C.

2B: The same process is performed in order to form a gel of 100 mg dipalmitoyl phosphatidyl choline and 10 mg dimyristoyl phosphatidyl glycerol.

2C: The same process is carried out in order to form a gel of 60 mg dipalmitoyl phosphatidyl choline and 6 mg dimyristoyl phosphatidyl glycerol.

### Example 3

180 mg dipalmitoyl phosphatidyl choline and 20 mg dimyristoyl phosphatidyl glycerol are combined with 30 mg coenzyme Q₁₀ in chloroform. The organic solvent is evaporated in vacuum and the remaining phosphatidyl Q₁₀ mixture is deposited as a thin film on a glass wall. 1 ml distilled water are added and the container is shaken on a shaker at low speed until a gel is formed. The gel is transferred into a syringe and stored at 4° C.

What is claimed is:

1-12. (canceled)

13. A phospholipid gel comprising a first phospholipid, a second phospholipid, and water, wherein the first phospholipid is a neutral phospholipid and the second phospholipid is a negatively charged phospholipid.

14. The phospholipid gel according to claim 13, wherein the first and second phospholipids are of natural origin, semi-synthetic origin, or synthetic origin.

15. The phospholipid gel according to claim 13, wherein the first and second phospholipids are selected from di(C₈-C₂₂ acyl) phosphatidyl choline and di(C₈-C₂₂ acyl) phosphatidyl glycerol.

16. The phospholipid gel according to claim 15, wherein the first and second phospholipids are dipalmitoyl phosphatidyl choline and dipalmitoyl phosphatidyl glycerol.

17. The phospholipid gel according to claim 13, wherein a total phospholipid concentration is within a range of 6-40% by weight.

18. The phospholipid gel according to claim 15, wherein the phosphatidyl choline and the phosphatidyl glycerol are present in a ratio within a range of 10:1 to 10:0.25.

19. The phospholipid gel according to claim 13 containing a pharmacologically active substance.

20. The phospholipid gel according to claim 19, wherein the pharmacologically active substance is a steroid; a non-steroidal antiphlogistic agent; an antibiotic; an antioxidant; or an antiepileptic agent.

21. The phospholipid gel according to claim 20, wherein the steroid is selected from the group consisting of cholesterol, hydrocortisone, and dexamethasone.

22. The phospholipid gel according to claim 20, wherein the non-steroidal antiphlogistic agent is selected from the group consisting of ibuprofen, diclofenac, flurbiprofen, and nabumetone.

23. The phospholipid gel according to claim 20, wherein the antibiotic is selected from the group consisting of tetracycline, a derivative of tetracycline, an aminoglycoside, a macrolid antibiotic, a nitroimidazole derivative, an antibiotic peptide, and an antibiotic oligonucleotide.

24. The phospholipid gel according to claim 23, wherein the aminoglycoside is gentamycine or neomycine; wherein the macrolid antibiotic is erythromycine; and wherein the nitroimidazole derivative is metronidazole or flucidic acid.

25. The phospholipid gel according to claim 20, wherein the antioxidant is selected from the group consisting of vitamin E and coenzyme Q₁₀

26. The phospholipid gel according to claim 20, wherein the antiepileptic agent is selected from the group consisting of valproic acid and salts of valproic acid.

27. The phospholipid gel according to claim 13 produced by shaking, vortexing, mixing by a stirrer, extrusion, or homogenization.

28. A method for moisturizing or calming normal or diseased skin or mucous membrane, the method comprising the step applying a substance containing the phospholipid gel according to claim 13.

29. A method of preparing medicaments or cosmetic substances for treating the skin, mucous membrane, natural or surgically generated body cavities or body compartments accessible by local or parenteral application, the method comprising the step of providing the phospholipid gel according to claim 13 as a carrier.

30. A method of stabilizing a solution, wherein the method comprises the step of adding the phospholipid gel according to claim 13 to the solution for solubilizing hardly soluble substances and/or preventing precipitation.
